(19) United States
(12) Patent Application Publication
(10) Pub. No.: US 2009/0060836 A1
(43) Pub. Date: Mar. 5, 2009

(54) DERIVATIVES OF 2-NITRO-1,3-IMIDAZOLE COUPLED TO AMINO ACIDS AND DEOXYRIBOSE USEFUL FOR THE DETECTION OF HYPOXIC BIOLOGICAL TISSUE

(75) Inventors: Hans-Juergen Machulla, Tuebingen (DE); Michael Uebele, Rottenburg (DE); Christiane Goerke, Rottenburg (DE)

Correspondence Address: KNOBBE MARTENS OLSON & BEAR LLP, 2040 MAIN STREET, FOURTEENTH FLOOR, IRVINE, CA, 92614, US

(73) Assignee: Eberhard-Karls-universitaet Tubingen Universitaetsklinikum, Tuebingen (DE)

(21) Appl. No.: 12/143,682

(22) Filed: Jun. 20, 2008

Related U.S. Application Data
(63) Continuation of application No. PCT/EP2006/012094, filed on Dec. 15, 2006.

(30) Foreign Application Priority Data
Dec. 21, 2005 (DE) DE102005063244.0

(51) Int. Cl. A61K 49/00; C07D 233/91; A61K 51/04; C07H 19/04

(52) U.S. Cl. 424/1.65; 548/327.5; 536/28.8; 424/9.1; 424/1.73

(57) ABSTRACT

The present invention relates to a 2-nitroimidazole derivative, a diagnostic pharmaceutical composition which contains such 2-nitroimidazole derivative, the use of the 2-nitroimidazole derivative for the detection of hypoxic biological tissue as well a method for the detection of hypoxic biological tissue by the use of the 2-nitroimidazole derivative.

### CROSS-REFERENCE TO RELATED APPLICATIONS

This application is a continuation of copending international patent application PCT/EP2006/012094 filed on Dec. 15, 2006, and designating the U.S., which was not published under PCT Article 21(2) in English, and claims priority of German patent application DE 10 2005 063 244.0 filed on Dec. 21, 2005, which is incorporated herein by reference.

### FIELD OF THE INVENTION

The present invention relates to a 2-nitroimidazole derivative, a diagnostic pharmaceutical composition containing such 2-nitroimidazole derivative, the use of such 2 nitroimidazole derivative for the detection of hypoxic biological tissue as well as a method for the detection of hypoxic biological tissue using the 2 nitroimidazole derivative.

### DESCRIPTION OF THE RELATED ART

Nitroimidazoles are generally known in the state of the art, for example from the WO 01/58434 A2 as well as from the chemical abstracts CA 143:281737, CA 143:281719, CA 127:136024, CA 140:195423, CA 138:69052, CA 137:365607. They are mainly described as chemotherapeutics and means against protozoans in intestinal infections by lamblias, amebic dysentery, trichomonosis as well as for infections by anaerobic bacteria. Nitroimidazole derivatives are also used as prophylaxis in surgical interventions in the large intestine and in the gynecology.

The measurement of oxygen deficiency in an organism, individual parts of the body or tissues, so-called hypoxia, is of extraordinary clinical relevance. It was found that tumour patients after an anti-cancer therapy are free of complaints over a longer time, however after a while they develop new tumours, very often only after one year, it comes to a so-called “recrudenscence”. Starting points for such a new formation of a tumour are hypoxic, i.e. oxygen-deficient tumour cells which have survived the therapy. Such hypoxic tumour cells can induce the new formation of tumours, the angiogenesis, and thereby help the tumour in growing.

Also the suffering from diabetes mellitus or an apoplexia very often results in hypoxic tissue areas of the affected patient. Hypoxic tissues are in parts largely constricted in their physiological function. Moreover, in the following very often in the affected areas it comes to necroses or to apoptosis with the consequence of an impairment of the whole organism.

Against this background especially in the clinical diagnostic there is a need to diagnose and localize a hypoxia. Only this establishes the possibility to detect e.g. a potential new formation of a tumour in due time and to counteract or even prevent a new formation of a tumour by the use of anti-carcinogenic measures. In the same time hypoxic tissue areas signalize e.g. disorders in the metabolism or the blood circulation which in case of a diagnosis in due time can be counteract.

At present hypoxic tissue areas are detected by labelled nitroimidazoles and their derivatives. It is known that such compounds accumulate in tissues in case of an oxygen deficiency. The known nitroimidazole derivatives diffuse into the biological cells. Inside the cells such compounds are reduced to radical anions by reducing enzymes, the reductases, which can there be found ubiquitously. At a normal concentration of oxygen such radical anions are again oxidized into the neutral molecule form since the redox potential of nitroimidazole and its derivatives towards oxygen is of a certain range, that they can again diffuse out of the tissue. At a lowered concentration of oxygen or hypoxia, respectively, in the biological cell there is a much higher probability that the radical anion as a highly reactive species will intracellularily collide with other molecules. This results in the case of hypoxia in a binding of the nitroimidazole derivatives to other molecules which in this manner accumulate intracellularily.

If such nitroimidazole derivatives are radioactively labelled, e.g. with fluorine 18, iodine 123 or iodine 124, a radioactive compound accumulates in the cell and is then measurable outside of the body by nuclear medical measurement procedures such as SPECT (single photon emission computed tomography) and PET (positron emission tomography) and can also be tracked in view of their reaction kinetics. One of the currently important representatives of such a nitroimidazole derivative is FMISO (fluoromisonida-zole):

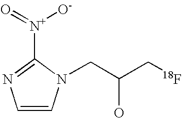

The nitroimidazoles and their derivatives which are used in the state of the art so far base on the above-mentioned concept according to which such compounds attain in the hypoxic tissue by mere diffusion. The involved disadvantage is that such tissue in which such nitroimidazoles or their derivatives have been accumulated, are of low contrast in imaging processes. For example biological tissue can only poorly be distinguished from blood. Such known nitroimidazoles or their derivatives are therefore poorly suited for a clinical routine use.

### SUMMARY OF THE INVENTION

One embodiment is a 2-nitroimidazole derivative comprising a vehicle which mediates the transport of the derivative into biological cells via cellular transport systems.

One embodiment is a 2-nitroimidazole derivative comprising a vehicle which mediates the transport of the derivative into biological cells via cellular transport systems, wherein the vehicle is selected from amino acids, 2′-deoxy-D-glucose and fatty acids.

Another embodiment is a 2-nitroimidazole derivative comprising a vehicle which mediates the transport of the derivative into biological cells via cellular transport systems, wherein the derivative comprises the following chemical structure:

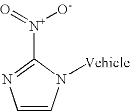

Another embodiment is a 2-nitroimidazole derivative comprising a vehicle which mediates the transport of the derivative into biological cells via cellular transport systems, wherein the vehicle comprises the following chemical structure:

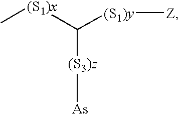

whereby S₁, S₂, and S₃ are selected from the group consisting of alkyl, alkenyl, aryl and arylalkyl groups; ether, ester and amid bridges; and linked, interrupted alkyl, alkenyl, aryl and arylalkyl groups; and

whereby x, y, z is an integer from 0 to 8, and

whereby Z is a leaving group for a nucleophilic substitution at aliphatic or aromatic carbon atoms or a detectable marker, and

whereby As is an amino acid unit which is linked via a C—C— or C—O-bond, or short peptides having a free amino acid function.

Another embodiment is a 2-nitroimidazole derivative comprising a vehicle which mediates the transport of the derivative into biological cells via cellular transport systems, wherein the vehicle comprises the following chemical structure:

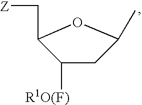

whereby Z is a leaving group for a nucleophilic substitution at aliphatic carbon atoms or a detectable marker, and

whereby R¹ is an H atom or an OH protecting group.

Another embodiment is a diagnostic pharmaceutical composition which comprises a 2-nitroimidazole derivative comprising a vehicle which mediates the transport of the derivative into biological cells via cellular transport systems and a pharmaceutical acceptable carrier.

Another embodiment is a method for detecting hypoxic biological tissue in a human or animal being, comprising the following steps:
1) provision of a 2-nitroimidazole derivative,
2) administration of the 2-nitroimidazole derivative into a human or animal being, or incubation of a tissue sample of the latter with the 2-nitroimidazole derivative,
3) measuring of any accumulation of the 2-nitroimidazole derivative in the human or animal being, or in the tissue sample of the latter, and
4) detection of hypoxic tissue in case of a positive result in step 3, wherein a 2-nitroimidazole derivative comprising a vehicle which mediates the transport of the derivative into biological cells via cellular transport systems is used.

### DETAILED DESCRIPTION OF THE PREFERRED EMBODIMENT

Against this background the problem underlying the present invention is to provide an improved nitroimidazole derivative by which the disadvantages of the state of the art can be avoided. In particular, a 2-nitroimidazole derivative should be provided by which hypoxic tissue areas in an organism can be detected in a reliable manner, whereby in imaging processes the contrast of the image between the accumulation and the distribution of the derivative inside the tissue should be considerably increased.

The problem is solved by the provision of a 2-nitroimidazole derivative comprising a vehicle which mediates the transport of the derivative into biological cells via cellular transport systems.

The inventors have surprisingly realized that by providing a vehicle at the 2-nitroimidazole derivative biological transport systems in the cellular membrane can be used to transport efficiently the derivative into the cell. In contrast to the known nitroimidazoles or derivatives, respectively, which merely reach the hypoxic tissue by passive diffusion, the 2-nitroimidazole derivative according to the invention is transported into the interior of the cell in a targeted manner by the use of cellular transport systems, whereby the concentration in the tissue is rapidly higher than in the blood.

Thus, with the 2-nitroimidazole derivative according to the invention hypoxic tissue areas can be better imaged than with the known nitroimidazole derivatives and consequently a more reliable diagnosis is enabled. Such a 2-nitroimidazole derivative according to the invention, which is transported in the cytoplasm in a targeted manner, provides a considerably increased imaging contrast in imaging processes, which enables a clinical routine use of the same.

According to the invention a “vehicle” refers to such a molecule which is permanently and stably bound to the 2-nitroimidazole, e.g. via a covalent bond. This vehicle mediates via an interaction with or via a binding to cellular system(s), respectively, such as e.g. amino acid transporters, nucleoside transport systems, a targeted translocation of the whole derivative into the interior of the biological cell. Cellular transport systems are well-known in the art; cf. Alberts et al. (2005), Lehrbuch der Zellbiologie [Text Book of Cell Biology], 3^{rd} edition, publisher Wiley-VCH.

It is preferred if the vehicle is selected from amino acids, preferably in their L-enantiomeric form, 2′-deoxy-D-glucose and fatty acids.

By this measure such vehicles are provided which use especially adequate and efficient transport systems, namely transporters for amino acids, nucleosides and fatty acids. Thereby e.g. aromatic amino acids such as tyrosine, attain into the cell via the cellular amino acid transporters, even if they comprise sterically large substituents, such as iodine or ethyl groups. Such substituted amino acids are not converted any further in the cell or do not enter the metabolism of the cell, respectively. Therefore, they provide also the option to transport, as a transport vehicle, the nitroimidazole function into the cell.

For these vehicle amino acids for the transport in the cell only the amino acid function is highly selective. This can be seen from the fact that only one pure enantiomeric form, e.g. L-tyrosine, is taken up into the cell. Except for proline which is only taken up by or transported into the cells, respectively, as a D-enantiomer, the remaining amino acids are taken up or transported, respectively, as a L-enantiomer.

Therefore, according to the invention the vehicle amino acid is bound to the 2-nitroimidazole function in such a manner that the amino acid function remains free and the amino acid is present as a pure enantiomer.

The provision of 2′-deoxy-D-glucose as vehicle has the advantage that the highly selective nucleoside transport system of the cell is used. Thereby, by the choice of the stereoisomers of the 2′-deoxy-D-glucose the selectivity can additionally optimized in an advantageous manner. That is why the coupling of a 2-nitroimidazole to a 2′-deoxy-D-glucose results in alpha- and beta-stereoisomers. These are highly selective since the nucleoside transporters transport exclusively the beta variant. For the alpha isomers it is known that to some extent they block the transporters and are not taken up into the cell.

As it was concluded from studies with thymidine derivatives, e.g. with 3′-deoxy-3′-fluorothymidine (FLT), a fluorinated fluorine derivative, slight changes of the deoxyribose are possible in an advantageous manner. For example the OH group of the C₃ atom can be substituted, e.g. with fluorine. This substitution blocks the chain formation in the DNA synthesis. FLT is only phosphorylated in the cell, this is rapidly reversible. Without the blockage of this 3′ side in case of the nitroimidazole derivatives hypoxia can be simulated by the incorporation of the vehicle (thymidine) into the DNA. Thereby the cellular uptake is not blocked, although the latter is quantitatively reduced.

According to the invention it is preferred if the 2-nitroimidazole derivative comprises the following chemical structure

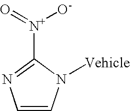

Thus, the 2-nitroimidazole derivative is realized in an advantageous manner.

It is further preferred if the vehicle comprises the following chemical structure:

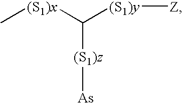

whereby S₁, S₂, S₃ are selected from alkyl, alkenyl, aryl, arylalkyl groups, ether, ester, amide bridges, linked, interrupted alkyl, alkenyl, aryl, arylalkyl groups, and whereby x, y, z is an integer from 0 to 8, and

whereby Z is a leaving group for a nucleophilic substitution on aliphatic or aromatic carbon atoms or an detectable marker, and

whereby As is an amino acid unit linked via a C—C— or C—O-bond, or also short peptides having a free amino acid function.

As it was realized by the inventors with this structure an 2-imidazole derivative according to the invention is realized in an especially advantageous manner, which can be transported into biological cells via cellular transport systems in a targeted manner, and is appropriate for the detection of hypoxic tissue.

The nitroimidazole derivative according to the invention further provides a precursor for a nucleophilic halogenation, for example a [¹⁸F]-fluorination. This precursor can be transferred into a correspondingly radioactively labelled compound, which is then detectable in the hypoxic tissue areas via nuclear medical methods.

According to the invention As encompasses natural or non natural amino acids in their D- and/or L-enantiomeric forms, as well as short peptides consisting of approximately 2 to 100 of such amino acids. The linkage of the amino acid unit As is realized in such a manner that the actual amino acid function is not involved in the linkage but remains free or is provided with protecting groups for a labelling/halogenation reaction, respectively, if a precursor is provided, which in turn release the amino acid function via a subsequent hydrolysis.

According to the invention it is preferred if Z is selected from the group consisting of: triflates, nosylates, tosylates, halogen atoms, activated nitro or trimethylammonium groups.

With this measure especially adequate and approved leaving groups for a nucleophilic substitution at aliphatic or aromatic carbon atoms are provided, to provide a precursor for the nucleophilic halogenation, e.g. [¹⁸F]-fluorination.

It is further preferred if Z as detectable marker is selected from the group consisting of: ¹⁸F, ¹²³I, ¹²⁴I.

With this measure suitable and approved markers are provided, that the labelled 2-nitroimidazole derivative according to the invention is reliably measurable outside of the body by means of nuclear medical measuring procedures, such as SPECT and PET, and can be monitored in view of its reaction kinetics. Therefore, hypoxic tissue areas can be detected in a reliable manner.

Alternatively it is preferred if the vehicle comprises the following chemical structure:

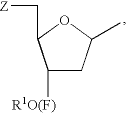

whereby Z is a leaving group for a nucleophilic substitution at aliphatic carbon atoms or a detectable marker, and whereby R¹ is an H atom or an OH protecting group.

According to the invention in this variant the carbon atom at position 3′ of the 2′-deoxy-D-glucose can be substituted by fluorine (F) instead by the R¹O group.

With this embodiment a 2-nitroimidazole derivative according to the invention is realized, which is coupled to a 2′-deoxy-D-glucose, so that a transport of the derivative using the nucleoside transport system into the cell can occur. According to the invention hereby alpha as well as beta isomers of the so-modified 2-nitroimidazole derivative are encompassed.

If Z is a leaving group for a nucleophilic substitution at aliphatic carbon atoms, the 2-nitroimidazole derivative provides also a precursor for the halogenation, e.g. for the [¹⁸F]-fluoride labelling, namely 1-[3-O-acyl-2-deoxy-5-O-toluolsulfonyl-β-D-ribofura-nosyl]-2-nitroimidazole (beta isomer) or 1-[3-O-acyl-2-deoxy-5-O-toluolsulfonyl-α-D-ribofuranosyl]-2-nitroimidazole (alpha isomer).

Appropriate leaving groups are: triflates, nosylates, tosylates, halogen atoms, activated nitro or trimethylammonium groups.

If Z is a detectable marker a 2-nitroimidazole derivative is provided which is detectable by nuclear medical measuring procedures and is directly suited for the clinical routine detection of hypoxic tissue areas.

Appropriate detectable markers are: ¹⁸F, ¹²³I, ¹²⁴I.

If an H atom is provided for R¹ and ¹⁸F for Z the compounds 1-[5-[¹⁸F]fluor-2,5-dideoxy-β-D-ribofuranosyl]-2-nitroimidazole (beta isomer) or 1-[5-[¹⁸F]fluor-2,5-dideoxy-α-D-ribofuranosyl]-2-nitroimidazole (alpha isomer), respectively, are provided.

With this variant according to the invention the OH protecting groups are preferably selected from the group consisting of: CH₃, CH₂OCH₃, CH₂OCH₂(C₆H₅), CH₂SCH₃, CH₂SCH₂(C₆H₅), CH₂OCH₂X (X=Cl, Br), CH₂COC₆H₄-4-Br, CH₂COC₆H₃-3,4-Cl₂, CH₂COC₆H₃-2,6-Cl₂, CH₂CH═CH₂, CH(CH₃)₂, c-C₆H₁₁, C(CH₃)₃, CH₂C₆H₅, 2,6-(CH₃)₂C₆H₃CH₂, 4-CH₃OC₆H₄CH₂, o-NO₂—C₆H₄CH₂, (CH₃)₂NCOC₆H₄CH₂, COCH₃, COC₆H₅, COCH₂C₆H₅, CO₂CH₃, COOCH₂CCl₃, CONHR(R=Ph, i-Bu).

With this measure approved OH protecting groups are provided in an advantageous manner, which are required for an ¹⁸F labelling reaction. They prevent an HF binding (in case of a free OH function) which would result in an escape of the fluoride from the reaction.

It is especially advantageous if the carbon atom at position 3′ of 2′-deoxy-D-glucose is substituted by fluorine (F) instead by the R¹O group.

Another subject-matter of the present invention relates to a diagnostic pharmaceutical composition which comprises the before-described nitroimidazole derivative as well as a pharmaceutical acceptable carrier and, if appropriate, further active agents and/or excipients.

Pharmaceutical acceptable carriers as well as active agents and excipients are generally known in the art and to a skilled person; cf. A. H. Kibbe, Handbook of Pharmaceutical Excipients, 3^{rd} edition, American Pharmaceutical Association and Pharmaceutical Press after Kibbe (2000). The content of this publication is incorporated herein by reference.

Furthermore, the present invention relates to a method for the detection of hypoxic biological tissue in a human or animal being, which comprises the following steps: provision of a before-described 2-nitroimidazole derivative, administration of the 2-nitroimidazole derivative into a human or animal being or incubation of a tissue sample of the latter with the 2-nitroimidazole derivative, measurement of any accumulation of the 2-nitroimidazole derivative in the human or animal being or the tissue sample of the latter, and detection of hypoxic tissue in case of a positive result in the step before.

It goes without saying that the previously mentioned features and those to be mentioned in the following cannot only be used in the combination which is indicated in each case, but also in other combinations or in isolated form, without departing the scope of the present invention.

In the following embodiments of the invention are shown which result in further advantages.

### Example 1

In the following an example of a synthesis of a 2-nitroimidazole derivative coupled to an amino acid is shown. As a coupling component e.g. a triol, here glycerol can be used. Many other variants of the reaction are conceivable. R₁ is any substituted benzaldehyde derivative for the masking of 1,3-diol functions. Z is a leaving group for a substitution reaction, e.g. -Br or -tosylate. R′ and R″ are any NH₂ or COOH protecting groups. L is any substituent. ¹⁸F is fluoride-18. The compound 1a shows the labelling precursor, the compound 2a the tracer.

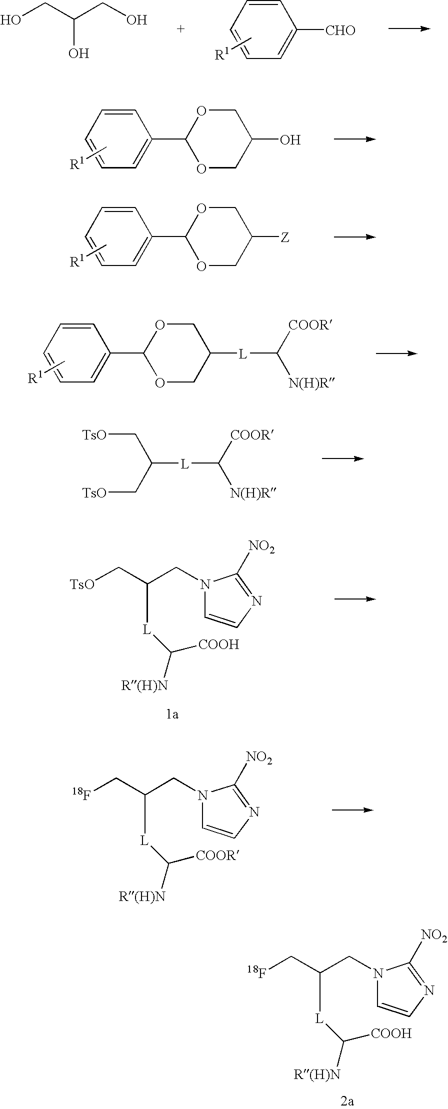

### Example 2

In the following a further example of a synthesis of a 2-nitroimidazole derivative coupled to deoxyribose is shown. The possible variants of the synthesis are numerous. If it is started with a fluorinated deoxyribose in the first step the fluorinated products (tracer) 5 and 6 are obtained via the labelling precursors 3 and 4.

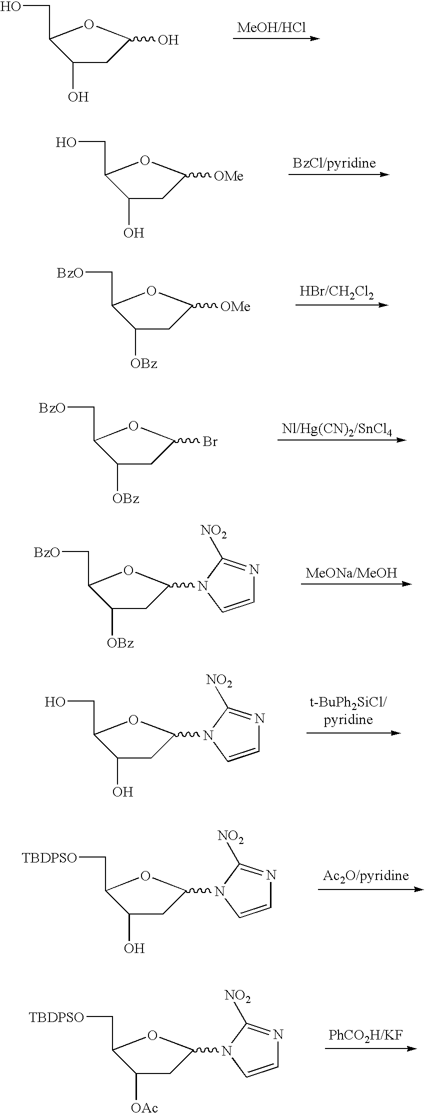

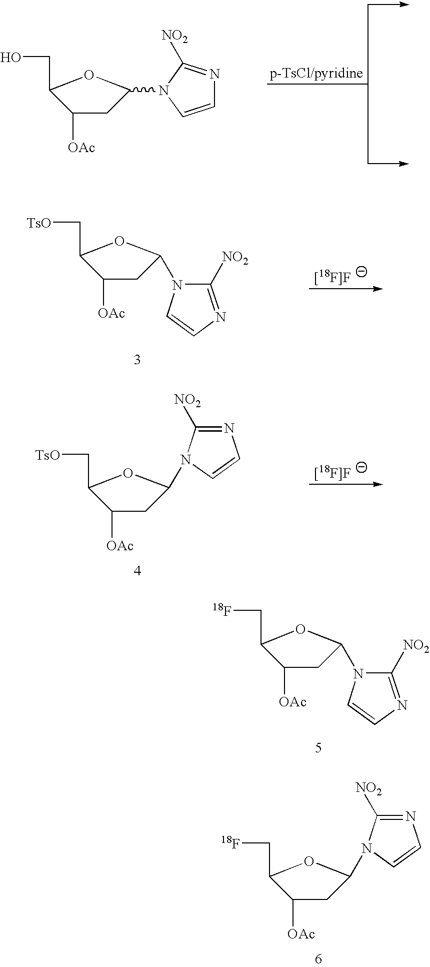

As end products an α/β-product mixture is obtained which can be chromatographically separated.

### Example 3

In the following an example of a synthesis of a 2-nitroimidazole derivative coupled to a fatty acid is shown (labelling precursor 7, tracer 8). The possible variants of the synthesis are numerous. Z corresponds to a leaving group for a nucleophilic substitution, R₁ is a carbonyl protecting group (CH₃, C₂H₅, CH₂OCH₃, CH₂SCH₃, CH₂OCH₂C₆H₅, CH₂CCl₃, C(CH₃)₃, CH₂C₆H₅, CH₂C₆H₂-2,4,6-(CH₃)₃) and Y can be a proton, a halogen atom, a hydroxyl function or an alkyl group. R² is an ether group (methyl, ethyl, etc.) which can be selectively cleaved off if an ester group is obtained. The groups S₁, S₂ and S₃ are selected from the alkyl, alkenyl, aryl, arylalkyl groups, ether, ester, amid bridges, linked, interrupted alkyl, alkenyl, aryl, arylalkyl groups; x, y, z is an integer from 0 to 8, OTs or TsO, respectively, is tosylate.

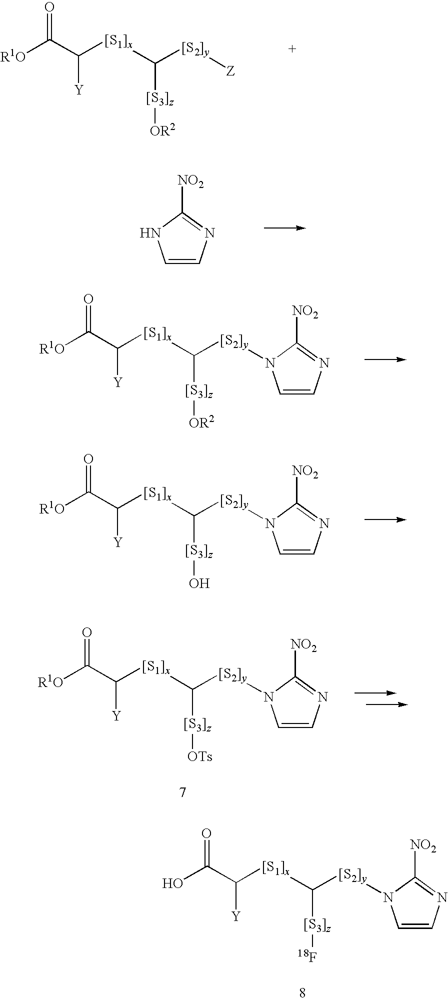

What is claimed is:

1. A 2-nitroimidazole derivative comprising a vehicle which mediates the transport of the derivative into biological cells via cellular transport systems.

2. The 2-nitroimidazole derivative of claim 1, wherein the vehicle is selected from amino acids, 2′-deoxy-D-glucose and fatty acids.

3. The 2-nitroimidazole derivative of claim 1, wherein the amino acids are in their L-enantiomeric form.

4. The 2-nitroimidazole derivative of claim 1, wherein the derivative comprises the following chemical structure:

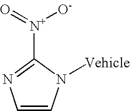

5. The 2-nitroimidazole derivative of claim 1, wherein the vehicle comprises the following chemical structure: 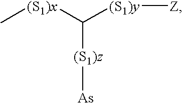
whereby S₁, S₂ and S₃ are selected from the group consisting of alkyl, alkenyl, aryl and arylalkyl groups; ether, ester and amid bridges; and linked, interrupted alkyl, alkenyl, aryl and arylalkyl groups; and
whereby x, y, z is an integer from 0 to 8, and
whereby Z is a leaving group for a nucleophilic substitution at aliphatic or aromatic carbon atoms or a detectable marker, and
whereby As is an amino acid unit which is linked via a C—C— or C—O-bond, or short peptides having a free amino acid function.

6. The 2-nitroimidazole derivative of claim 5, wherein Z is selected from the group consisting of: triflates, nosylates, tosylates, halogen atoms, activated nitro and trimethylammonium groups.

7. The 2-nitroimidazole derivative of claim 5, wherein Z is selected from the group consisting of: ¹⁸F, ¹²³I and ¹²⁴I.

8. The 2-nitroimidazole derivative of claim 1, wherein the vehicle comprises the following chemical structure: 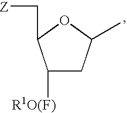
whereby Z is a leaving group for a nucleophilic substitution at aliphatic carbon atoms or a detectable marker, and
whereby R¹ is an H atom or an OH protecting group.

9. The 2-nitroimidazole derivative of claim 8, wherein Z is selected from the group consisting of: triflates, nosylates, tosylates, halogen atoms, activated nitro and trimethylammonium groups.

10. The 2-nitroimidazole derivative of claim 8, wherein Z is selected from the group consisting of: ¹⁸F, ¹²³I and ¹²⁴I.

11. The 2-nitroimidazole derivative of claim 8, wherein the OH protecting group is selected from the group consisting of: CH₃, CH₂OCH₃, CH₂OCH₂(C₆H₅), CH₂SCH₃, CH₂SCH₂(C₆H₅), CH₂OCH₂X (X=Cl, Br), CH₂COC₆H₄-4-Br, CH₂COC₆H₃-3,4-Cl₂, CH₂COC₆H₃-2,6-Cl₂, CH₂CH═CH₂, CH(CH₃)₂, c-C₆H₁₁, C(CH₃)₃, CH₂C₆H₅, 2,6-(CH₃)₂C₆H₃CH₂, 4-CH₃OC₆H₄CH₂, o-NO₂—C₆H₄CH₂, (CH₃)₂NCOC₆H₄CH₂, COCH₃, COC₆H₅, COCH₂C₆H₅, CO₂CH₃, COOCH₂CCl₃ and CONHR(R=Ph, i-Bu).

12. A diagnostic pharmaceutical composition which comprises the 2-nitroimidazole derivative of claim 1 and a pharmaceutical acceptable carrier.

13. A method for detecting hypoxic biological tissue in a human or animal being, comprising the following steps:
1) provision of a 2-nitroimidazole derivative,
2) administration of the 2-nitroimidazole derivative into a human or animal being, or incubation of a tissue sample of the latter with the 2-nitroimidazole derivative,
3) measuring of any accumulation of the 2-nitroimidazole derivative in the human or animal being, or in the tissue sample of the latter, and
4) detection of hypoxic tissue in case of a positive result in step 3, wherein a 2-nitroimidazole derivative of claim 1 is used.
